(19) United States
(12) Patent Application Publication
(10) Pub. No.: US 2005/0147665 A1
(43) Pub. Date: Jul. 7, 2005

(54) Pharmaceutical and nutritional compositions

(75) Inventors: David Frederick Horrobin, Stirling (GB); Christina Gouaille, Helisingborg (SE)

Correspondence Address: JACOBSON HOLMAN PLLC, 400 SEVENTH STREET N.W., SUITE 600, WASHINGTON, DC, 20004, US

(73) Assignee: Laxdale Limited

(21) Appl. No.: 10/995,533

(22) Filed: Nov. 24, 2004

Related U.S. Application Data
(63) Continuation of application No. 09/615,736, filed on Jul. 13, 2000, now abandoned.

(30) Foreign Application Priority Data
Jul. 14, 1999 (GB) 9916536.7

(51) Int. Cl. A61K 31/714; A61K 31/202; A61K 31/4415; A61K 31/525; A61K 9/64

(52) U.S. Cl. 424/456; 514/52; 514/251; 514/560; 514/350

(57) ABSTRACT

The combined application of at least one essential fatty acid of the n-6 or n-3 series, optionally together with further essential fatty acid(s) of the n-6 or n-3 series, together with one or more homocysteine lowering agent. The homocysteine lowering agent is selected from vitamin B12, folic acid, a compound related to folic acid with similar biological activity and vitamin B6.

In the past ten years evidence has accumulated which indicates that elevated blood and tissue levels of homocysteine indicate an increased risk of all forms of cardiovascular disease (including coronary heart disease, venous and arterial thrombosis and peripheral vascular disease) (M den Heijer et al, Arterioscler Thromb Vasc Biol 18: 356-361, 1998: M den Heijer et al Thromb Haemostas 80: 874-877, 1998: L M Taylor et al, J Vasc Surgery 29: 8-21, 1999: N J Wald et al, Arch Intern Med 158: 862-867, 1998: H Refsum et al, Ann Rev Med 49: 31-62, 1998), of cerebrovascular disease and stroke (J-H Yoo et al, Stroke 29: 2478-2483, 1998: C D A Stehouwer et al, Arterioscler Thromb Vasc Biol 18: 1895-1901) of diabetes, pre-diabetes (insulin resistance or syndrome X) and its various complications including vascular disease, kidney disease, nerve damage and eye damage (S Neugebauer et al, Lancet 352: 454, 1998: A K Aarsand et al, J Internal Med 244: 169-174, 1998: E J Giltay et al, Atherosclerosis 139: 197-198, 1998: E Okada et al, Diabetes Care 22: 484-490, 1999), of a range of psychiatric disorders including depression and schizophrenia (E Susser et al, Biol Psychiatry 44: 141-143, 1998: T Arinami et al, Am J Med Genetics 74: 526-528, 1997: C Gomes-Trolin et al, J Neural Trans, 105: 1293-1305, 1998: B Regland et al, J Neural Transm 98: 143-152, 1994: J E Albert et al, Nutrition Rev 55: 145-149, 1997: T Bottiglieri, Nutrition Rev 54: 382-390, 1996), of neurological disorders including Alzheimer's disease and other dementias (E Jensen et al, Arch Gerontol Geriatr 26: 215-226, 1998: R Clarke et al, Arch Neurol 55: 1449-1455, 1998: M Lehmann et al, Dementia 10: 12-20, 19.99), multiple sclerosis (STFM Frequin et al, J Neurol 240: 305-308, 1993: G A Qureshi et al, Biogenic Amines 12: 353-376, 1996) and Parkinson's disease, of kidney disorders and kidney failure (T Tamura et al, Am J Kidney Dis 32: 475-481, 1998: A Vychytil et al, Kidney Int 53: 1775-1782, 1998) of inflammatory disorders, including inflammatory bowel diseases and arthritis (S L Morgan et al, J Rheumatol 25: 441-446, 1998: M Cattaneo et al, Netherl J Med 52: S1-61, 1998), of ear and eye disorders including age-related macular degeneration, age-related hearing loss and tinnitus (DK Houston et al, Am J Clin Nutr 69: 564-71, 1999), of cancers (DG Weir et al, Am J Clin Nutr 68: 763-4, 1998: E Giovannucci et al, Ann Intern Med 129: 517-524, 1998) and of all-cause mortality (E K Hoogeveen et al, Netherlands J Med 52: S1-61, 1998). Homocysteine levels may also be elevated during obesity and particularly during its treatment (B F Henning et al, Res Exp Med 198: 37-42, 1998). Homocysteine-lowering nutrients may also be of value in the treatment of pain (J Leuschner, Arzneim-Forsch 42: 114-115, 1992) and during pregnancy for the prevention of congenital disorders such as spina bifida and of pregnancy problems such as pre-eclampsia or fetal growth restriction (M Leeda et al, Am J Obstet Gynecol 179: 135-139, 1998). The mechanism of these widespread associations between elevated homocysteine and disease remains unknown but is likely to be something which operates at a fundamental biochemical level in many different tissues. One strong candidate is excessive oxidation promoted by homocysteine and its metabolites leading to changes in the functions of proteins and lipids (P B Young et al, Atherosclerosis 129: 67-71, 1997). The endothelium may be particularly vulnerable and since the endothelium is important in every tissue of the body this could provide a basis for the extraordinary range of pathology which is associated with elevated homocysteine (J C Chambers et al. Circulation 99: 1156-1160, 1999).

The main determinants of elevated homocysteine levels are deficits of folic acid and of vitamin B12 and, to a lesser extent, of pyridoxine and related substances with vitamin B6 activity. Homocysteine is mainly metabolised by conversion to methionine, which can then be used to make S-adenosyl-methionine which is used as a methyl donor in many different essential reactions, including the regulation of DNA and RNA functions and the syntheses of phospholipids, neurotransmitters and complex carbohydrates. The conversion of homocysteine to methionine is catalysed by the enzyme methionine synthetase: methyl-cobalamin, one of the forms of vitamin B12, plays a critical role in this reaction. A required co-factor for the enzyme is folic acid in the form of methyl-tetrahydrofolate. In the course of the reaction, a methyl group is transferred from 5-methyltetrahydrofolate to homocysteine, so producing tetrahydrofolate and methionine. Adequate intake and absorption of both folic acid and vitamin B12 are therefore required to keep homocysteine levels low and to ensure proper methylation reactions.

A secondary route for the metabolism of homocysteine involves its conversion to cystathionine and then to cysteine in two separate reactions, both of which require vitamin B6 as a co-factor. Inadequate availability of pyridoxine or related molecules may therefore make a contribution to elevated homocysteine levels.

Optimal control of homocysteine metabolism therefore requires optimal body levels of vitamins B12 and B6 and also of folic acid or methyltetrahydrofolate or any other related substance which can provide folate. Vitamin B6 must be provided at a dose of at least 2 mg per day, and preferably 5 mg to 200 mg per day. Vitamin B12 is normally provided by injection but can be given by mouth, even in those who lack the gastric intrinsic factor required for efficient absorption from the gut. Daily oral doses of vitamin B12 of at least 200 μg, and preferably 500 to 10,000 μg are required to ensure adequate tissue levels in those such as the elderly in whom B12 absorption may not be fully normal. The vitamin B12 may be provided as cyanocobalamin or hydroxocobalamin or any other biologically active form of the vitamin. Hydroxocobalamin is the preferred form since it is relatively stable and does not act as a cyanide donor. Folic acid should be provided in a dose of at least 200 μg/day and preferably more than 500 μg/day. The best results in control of elevated homocysteine will be obtained by the appropriate oral administration of all three vitamins. Appropriate daily doses applicable to most people would be 1 mg to 5 mg of B12, preferably as hydroxocobalamin, 0.5 to 5 mg of folic acid, and 2 mg to 20 mg of pyridoxine.

Essential fatty acids are another class of essential nutrients, so-called because they cannot be made within the body but have to be provided in the diet. There are two types of EFAs, n-3 (or omega-3) and n-6 (or omega-6) which are not interchangeable. The main parent EFA of the n-6 group is linoleic acid, while the main parent fatty acid of the n-0.3 group is alpha-linolenic acid (FIG. 1). Although linoleic and alpha-linolenic acids are the most important EFAs in the diet, it is their metabolites which play the most important roles in the body. Although the metabolites cannot be synthesised de novo, they can be made from the parent EFAs by the pathways shown in FIG. 1. Particularly important members of the EFA families in terms of biological effects are dihomogammalinolenic acid (DGLA), arachidonic acid (AA), eicosapentaenoic acid (EPA) and docosahexaenoic acid (DHA).

Just as elevated levels of homocysteine have been associated with a remarkably wide range of illnesses, so low levels of essential fatty acids, and particularly low levels of the metabolites DGLA, AA, EPA and DHA, have also been associated with a wide and very similar range of illnesses. The illnesses in which reduced levels of these fatty acids have been found include cardiovascular diseases, cerebrovascular diseases, thrombotic diseases, psychiatric diseases such as schizophrenia, depression and bipolar disorder, inflammatory diseases such as various forms of arthritis, eczema, asthma and inflammatory bowel disease, diabetes and its complications, kidney disease, neurodegenerative diseases like Alzheimer's disease and other dementias and Parkinson's disease, kidney diseases, many forms of cancer, and disorders of the reproductive system including male and female infertility and disorders of the breast and prostate (D F Horrobin, ed, Omega-6 Essential Fatty Acids: Pathophysiology and Roles in Clinical Medicine, Wiley-Liss, New York, 1990: D F Horrobin and C N Bennett, Prostaglandins Leukotr Essential Fatty Acids, 60: in press, 1999: A Leaf et al, World Rev Nutr Diet 83: 24-37, 1998: DF Horrobin, Prostaglandins Leukotr Essential Fatty Acids, 53: 385-396, 1995).

Many studies have been performed in which EFAs have been used in attempts to treat diseases, including cardiovascular and cerebrovascular disorders, psychiatric and neurological disorders, renal disorders, inflammatory disorders of the skin, joints, respiratory and gastrointestinal systems, cancers and many other conditions. The EFAs which have been used have been particularly gamma-linolenic acid (GLA), DGLA, AA, EPA, and DHA, but also alpha-linolenic acid, linoleic acid and stearidonic acid. On the whole the results have shown beneficial effects but, equally, the effects have often been less than those hoped for by the authors.

Studies have also reported the levels of EFAs and the levels of homocysteine in patients or subjects: for example, in patients with end-stage renal disease, there has been reported an inverse relationship between the levels of homocysteine and the levels of DGLA, AA, EPA and DHA. Lowering homocysteine by treatment with folic acid produced elevations of these four fatty acids which were significant in the cases of DGLA and AA, but not significant in the cases of EPA and DHA (S Hirose et al. Jap J Nephrol 1998; 40: 8-16). Similarly, in rats made folic acid deficient, homocysteine levels became elevated and at the same time plasma levels of AA, EPA and DHA fell, the last two significantly so (P Durand et al, Atherosclerosis 1996; 121: 231-243). In another study., human maternal plasma homocysteine levels were related to fatty acid levels in the red cells of the babies. There was a strong inverse relationship between maternal homocysteine and baby DHA (H Bohles et al. Eur J Pediatrics 158: 243-246, 1999).

The present invention is based on the inventors' observation that there may be a close relationship between the elevation of homocysteine and the deficits of EFAs, especially of AA, EPA and DHA. EFAs, with their multiple double carbon-carbon Bonds, are highly susceptible to oxidation. Homocysteine and its metabolites could be promoting EFA-oxidation to reduce EFA levels.

Administration of the EFAs used in attempts to treat diseases may be being countered by their ongoing oxidation as a result of elevated levels of homocysteine. Thus the changes in EFA levels and the therapeutic effects of the EFAs will be less than expected. Moreover, since some of the oxidised EFA metabolites can be toxic, the desirable effects may be counteracted by undesirable ones.

Equally, attempts to lower homocysteine by means of folic acid, vitamin B12 or vitamin B6, either alone or in combination have often had desirable effects, but sometimes those desirable effects have been less than expected. This can be explained if some of the toxicity of homocysteine is attributable to loss of EFAs. Correction of elevated homocysteine will prevent the ongoing damage to the EFAs. However, since the EFAs cannot be synthesised de novo by the body, controlling homocysteine will do nothing to increase the supply of EFAs to help replace those which have been lost.

The following invention thus provides the combined application of one or more EFAs, together with one or more homocysteine lowering agents, for use in therapy of any disorder, but particularly of those disorders discussed earlier in this specification. It is preferred that the EFAs are administered in a formulation which has no significant amounts of other micro-nutrients; preferably the active ingredients of the formulation consist essentially wholly of the selected EFA(s) and the homocysteine lowering agent(s).

The homocysteine lowering agent(s) are preferably selected from Vitamin B12, folic acid, a compound related to folic acid and with similar biological activity and Vitamin B6. All four of these homocysteine-lowering agents can be administered together with the EFA, or any two or three of them. For example, it may not be appropriate to administer both folic acid and a compound related to folic acid. The folic acid could be administered with vitamin B12 or vitamin B6 or both. The other option is to choose just one of the homocysteine-lowering agents.

The most preferred EFAs used in the formulations of the present inventions are eicosapentaenoic acid (EPA) and arachidonic acid (AA). The EPA can be in the form of pure tri-EPA triglyceride or, more preferably, the ethylester. For any of the EFAs selected, the EFA may be in the purified or partly purified form, though preferably the purified form.

The formulations of the present invention are set out in the attached claims.

The lowering of homocysteine will prevent ongoing damage to the EFAs, and so make the desirable results of EFA administration more likely. Equally, the provision of EFAs will help to replenish fatty acids lost through elevated homocysteine levels, and so make a desirable response to lowering homocysteine more likely.

EFAs and homocysteine lowering nutrients have been naturally coadministered in the form of human and artificial milks, eggs and of other nutrient complete foods. However, they have not previously been administered in pharmaceutical or nutritional supplement dose forms, nor in the doses likely to be required for therapeutic as opposed to nutritional effects. In particular, oral administration of vitamin B12 in relatively high doses has rarely been employed, neither natural or artificial milks, nor multinutrient mixes for oral or enteral administration, contain levels of vitamin B12 which are anywhere close to 200 μg/day. Similarly, these foods contain levels of folic acid and of vitamin B6 which are far below 100 μg/day for folic acid and 1.5 mg per day vitamin B6.

For example, dried milk which is the complete food richest in these nutrients contains only 0.23 mg vitamin B6, 2.0 μg vitamin B12 and 40 μg folic acid per 100 g [The Composition of Foods, A A Paul and D A T Southgate, HMSO, London 1988]. 100 g of dried milk products provides about 500 calories and so it would be impossible to consume more that about 500 g/day of dried milk. Even this large amount would only provide 1.15 mg vitamin B6 and 10.0 μg vitamin B12.

The EFAs in the compositions and uses of the present invention may be in any form which leads to a rise in the level of the relevant EFA molecule in the plasma or in cell membranes. Appropriate forms include mono-, di- and triglycerides, phospholipids, esters of any form, including ethyl, propanediol or any other appropriate form of ester, amides, salts, including lithium, sodium and potassium salts, and any other compounds which, following oral, parenteral or topical administration lead to an increase in blood or tissue levels of the EFAs concerned. Particularly appropriate forms which are known to be highly compatible with administration to the human or animal body are triglycerides and ethyl esters, for example of GLA, DGLA, AA, EPA or DHA. The EFAs may be administered in doses of from 10 mg to 100 g. per day, preferably 50 mg to 20 g per day, and very preferably 100 mg to 5 g/day. The EFAs may be provided in the form of natural oils, partially or completely purified natural oils in which the other components have been removed, or chemically derivatised pure or partially purified lipid forms. The EFA component of the formulation must contain at least 5% of the relevant EFA or EFA derivative, preferably more than 15%, and very preferably more than 30%, 50%, 90% or 95%.

The homocysteine-lowering agents used in the compositions and uses of the present invention are selected from vitamin B12, folic acid or a related compound with similar biological activity and vitamin B6. The preferred form of vitamin B12 is hydroxocobalamin, though cyanocobalamin or any other biologically active form of the vitamin may be used. If present, more than 10 μg/day vitamin B12 is required. The preferred dosage is at least 200 μg, preferably 500-10,000 μg, still preferably 1 mg-5 mg per day. Folic acid may be used as it is or in the form of methyltetrahydrofolate or any other related substance which can provide folate. The preferred dosage is at least 200 μg, preferably more than 500 μg and still preferably 0.5-5 mg per day. Vitamin B6 may be used in the form of pyridoxine. If present, at least 1.5 mg/day vitamin B6 is required. The preferred dose is at least 2 mg, preferably 5-200 mg, still preferably 2-20 mg per day. Overall, it is preferred that at least 200 μg/day homocysteine lowering agent is required, whatever the identity of the said agent(s).

The EFAs and homocysteine-lowering nutrients may be mixed together in powders or liquids, may be administered together in tablets, hard or soft gelatin capsules, microcapsules or any other appropriate dosage form known to those skilled in the art. The EFAs and the homocysteine-lowering nutrients may also be given in separate dosage forms but provided together in a single pack with instructions for daily administration of both components. The formulations may comprise conventional diluent and/or excipients and flavouring agents may be added.

One of the problems of using EFAs either in nutrition or in therapy is that they are easily oxidised within the body to a wide range of products, some of which may be harmful. The body has a system of antioxidant devices to deal with this, but not every individual may have adequate antioxidant defences. This is because several of the key antioxidants are essential nutrients which must be provided in the diet and not all diets are adequate. It is therefore advantageous to provide with the formulations one or more antioxidants. Antioxidants of particular value are vitamin E in any of its natural or artificial forms, coenzyme Q in any of its natural or artificial forms, alpha-lipoic acid in any of its natural or artificial forms and vitamin C in any of its natural or artificial forms. When the antioxidant component is required, it may include any one or any combination of these agents. If present, the dosage of antioxidant is preferably from 1 mg to 5000 mg per day.

### BRIEF DESCRIPTION OF THE FIGURES

FIG. 1. The n-3 and n-6 series of essential fatty acids

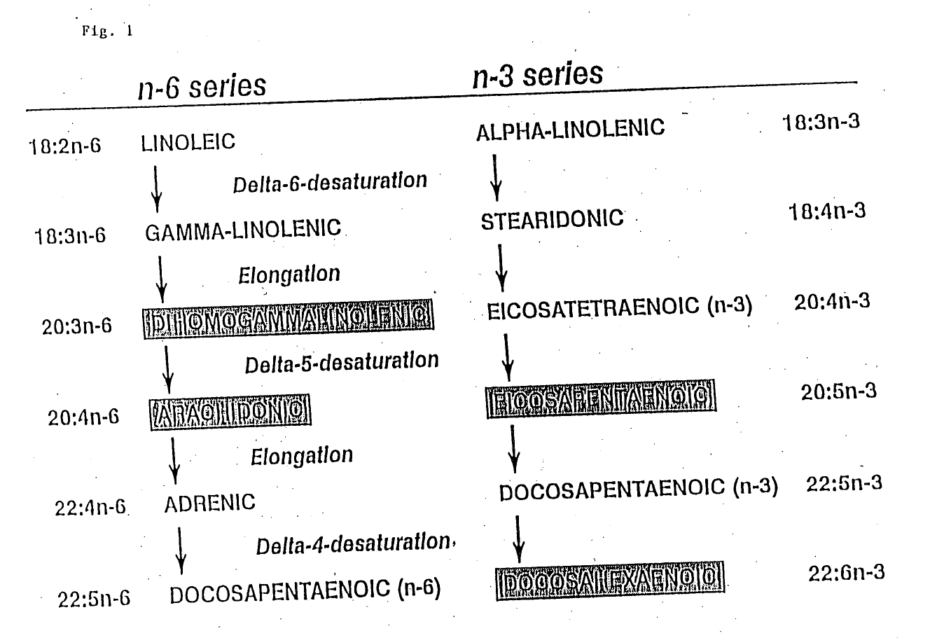

### EXAMPLES

1. Hard or soft gelatin capsules containing 500 mg of ethyl-eicosapentaenoate or of eicosapentaenoic acid triglyceride, together with 1 mg of hydroxocobalamin, 1 mg of folic acid and 2 mg of pyridoxine, to be taken two to four times a day.

2. A formulation as in 1 but in which the eicosapentaenoate is first microencapsulated with any appropriate microencapsulating agent and then tabletted with the other ingredients.

3. A solution for oral administration in which 500 mg of an eicosapentaenoate derivative, 1 mg of folic acid, 1 mg of hydroxocobalamin and 5 mg of pyridoxine are present in 5 ml with appropriate flavouring.

4. An emulsion for parenteral administration in which 500 mg of the eicosapentaenoate derivative is emulsified in a total volume of 10 ml, which includes in solution 1 mg of hydroxocobalamin, 1 mg of folic acid and 5 mg of pyridoxine.

5-8. As examples 1 to 4 but in which the EFA is selected from arachidonic acid, gamma-linolenic acid, dihomogammalinolenic acid, stearidonic acid, eicosapentaenoic acid, docosapentaenoic acid, docosahexaenoic acid, linoleic acid or alpha-linolenic acid or their derivatives.

9-12. As examples 1 to 4, but in which two or three EFAs selected from the list in 1-8 are coadministered to give a total of 500 mg of EFA per oral encapsulated or tabletted dosage form, per 5 ml solution, or per 10 ml parenteral emulsion.

13-24. As 1-12 but in which the only homocysteine-lowering component provided is vitamin B12.

25-36. As 1-12 but in which the only homocysteine-lowering component provided is folic acid.

37-48. As 1-12 but in which the only homocysteine-lowering component provided is vitamin B6.

49-96. As 1-48 in which one or more antioxidants selected from vitamin E, coenzyme Q, alpha-lipoic acid and vitamin C is added to the formulation. Vitamin E, coenzyme Q, alpha-lipoic acid and vitamin C may be used in doses of from 1 mg to 5000 mg per day,

1-15. (canceled)

16. A formulation consisting essentially of
eicosapentaenoic acid (EPA) or derivative;
vitamin B 12; folic acid, at a maximum daily dose of 5 mg; and vitamin B6, at a maximum daily dose of 20 mg; and,
optionally, at least one antioxidant.

17. A method for treating a subject suffering from one or more conditions selected from the group consisting of: a psychiatric disorder; schizophrenia, a schizotypal disorder and other schizophrenia form disorder, bipolar disorder (mania, or manic depression), depression; a panic disorder, an anxiety disorder, a sleep disorder; a social phobia, or a neurological disorder, a neurodegenerative disorder, Alzheimer's disease, dementia, Parkinson's disease, multiple sclerosis, and Huntington's disease, which comprises administering to the subject the formulation of claim 16.
